(19) United States
(12) Patent Application Publication
(10) Pub. No.: US 2018/0169085 A1
(43) Pub. Date: Jun. 21, 2018

(54) Combination Therapy for Hemorrhagic Injury

(71) Applicant: Augusta University Reserch Institute, Inc., Augusta, GA (US)

(72) Inventor: Raghavan Pillai Raju, Augusta, GA (US)

(21) Appl. No.: 15/846,803

(22) Filed: Dec. 19, 2017

Related U.S. Application Data
(60) Provisional application No. 62/436,178, filed on Dec. 19, 2016.

(51) Int. Cl. A61K 31/455; A61K 31/19; A61K 31/05; A61K 9/20; A61P 43/00

(52) U.S. Cl. CPC A61K 31/455; A61K 31/19; A61P 43/00; A61K 9/20; A61K 31/05

(57) ABSTRACT

It has been discovered that the combination of resveratrol, dichloroacetate and niacin has a synergistic effect when used to treat hemorrhagic injury. One embodiment provides a pharmaceutical composition containing an effective amount of resveratrol, dichloroacetate and niacin to improve cardiac function following severe hemorrhage versus a control that did not received the combination treatment. Resveratrol, dichloroacetate and niacin can each independently be present in the pharmaceutical composition in an amount from 2 mg/kg body weight to 10 mg/kg body weight. In one embodiment, resveratrol, dichloroacetate and niacin are each present in an amount of 2 mg/kg body weight. The pharmaceutical formulation can be formulated as one unit dose containing all three compounds. In other embodiments, each compound is formulated as a separate pharmaceutical composition that can be administered to a subject in need thereof simultaneously, in alternation, or in combination.

### CROSS-REFERENCE TO RELATED APPLICATIONS

This application claims benefit of and priority to U.S. Provisional Patent Application No. 62/436,178 filed on Dec. 19, 2016, which is incorporated herein in its entirety.

### STATEMENT REGARDING FEDERALLY SPONSORED RESEARCH

This invention was made with government support under GM101927-04 awarded by the National Institutes of Health, respectively. The government has certain rights in the invention.

### TECHNICAL FIELD OF THE INVENTION

The invention is generally related to pharmaceutical formulations for treating hemorrhagic injury or hemorrhagic shock.

### BACKGROUND OF THE INVENTION

Hemorrhagic injury (HI) is a leading cause of death in people under the age of 45 and accounts for almost half of trauma-related deaths. Hemorrhagic shock leads to whole body hypoxia, nutrient deprivation and dysregulation of critical biochemical pathways that may result in multiple organ dysfunction syndrome and death. Mitochondrial functional decline is a hallmark of hemorrhagic shock and enhanced mitochondrial function is known to contribute to better outcome following HI in animal models (Khoshmohabat, H., Trauma, 21(1):e26023 (2016); Perel, P., et al., BMJ, 345:e5166 (2012); Gonzalez, E., et al., Scand J Surg, 103(2):89-103 (2014)). Management of hemorrhagic shock necessitates timely fluid resuscitation. Though optimal resuscitation strategy remains controversial, hemorrhage control and resuscitation are high priorities in trauma care in civilian as well as combat situations (Shrestha, B., et al., J Trauma Acute Care Surg, 78(2):336-341 (2015); Percival, T. J., et al., Br J Surg, 99 Suppl 1:66-74 (2012); Rappold, J. F., et al., Transfusion, 53 Suppl 1:96S-99S (2013)).

Hypotensive volume replacement permits limited tissue reperfusion and continued metabolic activities to maintain cell viability. However, there is a lack of consensus on the use of any specific resuscitation strategy or adjuncts to resuscitation.

Timely hemostasis, volume replacement and efforts to maintain of cellular homeostasis can improve survival following HI. Hemostasis may be achieved by physical means or by promoting coagulation through pharmaceutical interventions. Although the hypotensive resuscitation strategy has gained strength, the volume and composition of the resuscitation fluid remains unsettled (Holcomb, J. B., et al., Shock, 35(2):107-113 (2011); Pusateri, A. E., et al., Shock, 39(2):121-126 (2013).

The hemostasis and fluid resuscitation procedures are expected to enable metabolic homeostasis spontaneously, though such a homeostatic balance is seldom realized. Cellular energetics are known to be an important determining factor in maintaining homeostatic balance. Therefore, adjuncts that facilitate metabolic activity required to maintain cell viability are important towards stabilizing wounded soldiers. Such adjuncts play an important role in hypotensive or aggressive resuscitation in maintaining cellular energetics and tissue function.

One of the major limitations in the treatment of hemorrhagic shock in the far forward management is administration of fluid resuscitation. Treatments that can prolong life in the absence of resuscitation fluid are important in such situations. Previous studies used an aggressive resuscitation strategy. There are now well standardized hemorrhagic injury (HI) models, in rat and mouse, having severe blood loss followed by shock and low volume resuscitation. These models allow one to screen compounds for their effect on survival. The models also allow one to test the effects of compounds on molecular mechanisms and organ function following HI.

The decreased oxygen and nutrient availability due to HI impairs mitochondrial function resulting in declined ATP production. The alteration of cellular energetics following hemorrhagic shock adversely affect cell survival and organ function. The mitochondrial oxidative damage by free radicals causes a vicious cycle that perpetuates more free radical production and further damage to mitochondria, leading to declining mitochondrial function (Vaidya, A. B., et al., Annu Rev Microbiol, 63:249-267 (2009); Wallace, D. C., Cold Spring Harb Symp Quant Biol, 74:383-393 (2009); Chen, Q., et al., Free Radic Biol Med, 40(6):976-982 (2006); Milei, J., et al., Cardiovasc Res, 73(4):710-719 (2007); Rushing, G. D., et al., Annals of Surgery, 247(6):929-937 (2008)). Therefore agents that alter mitochondrial function can have profound influence on outcome following HI.

Therefore, it is an object of the invention to provide compositions and methods for treating HI that modulate cellular energetics.

It is another object of the invention to provide combination therapies for the treatment of HI.

Still another object of the invention provides compositions and methods for improving organ function following severe hemorrhage.

### SUMMARY OF THE INVENTION

It has been discovered that the combination of resveratrol, dichloroacetate and niacin has a synergistic effect when used to treat hemorrhagic injury. Therefore, one embodiment provides a pharmaceutical composition containing an effective amount of resveratrol, dichloroacetate and niacin to improve organ function following severe hemorrhage versus a control that did not received the combination treatment. Exemplary organs to be improved include, but are not limited to the heart, brain, lung, and central nervous system. Resveratrol, dichloroacetate and niacin can each independently be present in the pharmaceutical composition in an amount from 2 mg/kg body weight to 10 mg/kg body weight. In one embodiment, resveratrol, dichloroacetate and niacin are each present in an amount of 2 mg/kg body weight. The pharmaceutical formulation can be formulated as one unit dose containing all three compounds. In other embodiments, each compound is formulated as a separate pharmaceutical composition that can be administered to a subject in need thereof simultaneously, in alternation, or in combination.

Another embodiment provides a method for improving organ function subsequent to severe hemorrhage in a subject by administering an effective amount of a combination of resveratrol, dichloroacetate and niacin to the subject in the absence of resuscitation fluids to improve cardiac function relative to a control. Still another embodiment provides a method of improving organ function subsequent to severe hemorrhage by administering the combination of resveratrol, dichloroacetate and niacin in conjunction with the administration of resuscitation fluids to the subject.

Still another embodiment provides a method for treating hemorrhagic shock by administering to a subject in need thereof a pharmaceutical composition comprising an effective amount of resveratrol, dichloroacetate and niacin to improve cardiac function relative to a control.

### BRIEF DESCRIPTION OF THE DRAWINGS

FIG. 1 is a graph of dP/dt (mmHg/sec) for untreated rats (SHAM), rats subjected to hemorrhagic injury and treated with vehicle (HI+Veh) and rats subjected to HI and treated with resveratrol (HI+RSV) or SRT1720.

FIG. 2 is a graph of cardiac ATP content (%) (relative to sham) for sham rats, rats subjected to HI, and rats subjected to HI and treated with RSV.

FIG. 3 shows Kaplan-Meier survival curves of percent survival versus days for rats treated with HI and vehicle (●) or rats treated with HI and RSV (□). HI+Veh (n=12) and HI+RSV (n=8) groups. p<0.05.

FIG. 4 shows Kaplan-Meier survival curves of percent survival versus days for rats treated with vehicle and HI (◯), HI and RSV (▪), or HI and SRT1720. HI+Veh, HI+RSV and HI+SRT1720 (n=5-6). *p<0.05 versus HI+Veh, both curves.

FIG. 5 shows Kaplan-Meier survival curves of percent survival versus time (min) for rats treated with HI and vehicle (●), HI and Dichloroacetate (DCA) 1 mg/kg (▪), HI and DCA 10 mg/kg (▴), or HI and DCA 25 mg/kg (▾), HI+Veh (n=7), HI+DCA 1 mg/kg (n=3), HI+DCA 10 mg/kg (n=5), HI+DCA 25 mg/kg (n=5); p<0.05 versus HI+Veh, all curves.

FIG. 6 shows Kaplan-Meier survival curves for rats treated with HI+Niacin (NA) 10 mg/kg (●), HI+NA 5 mg/kg (▪), HI+NA 1 mg/kg (▴), or HI+Veh (▾). HI+Veh (n=3), HI+Niacin 10 mg/kg (n=6), HI+Niacin 5 mg/kg (n=6) and HI+Niacin 1 mg/kg (n=4). p<0.05 versus HI+Veh, all curves.

FIG. 7 shows Kaplan-Meier survival curves for rats treated with HI+RSV+DCA+Niacin (NA) (2 mg/kg) (●), HI+Veh (▪), or HI+RSV 2 mg. HI+Veh (n=4), HI+RSV+DCA+NA 2 mg/Kg (n=2) and HI+RSV 2 mg/Kg (n=2).

### DETAILED DESCRIPTION OF THE INVENTION

### I. Definitions

The term “hemorrhagic shock” refers a life-threatening condition involving insufficient blood flow to the body tissues. Hemorrhagic shock is caused by excessive bleeding which reduces the blood volume.

The term “severe hemorrhage” refers to excessive bleeding that leads to hypotensive shock.

### II. Combination Therapy for Treating HI

### A. Niacin

The activity of SIRT1 is regulated by its cofactor NAD+. Therefore, agents that metabolize to NAD+ are expected to activate SIRT1. One of the metabolic products of niacin, or nicotinic acid, is NAD+. Niacin also has anti-inflammatory effect by signaling through its receptor GPR109a. Niacin is believed to have SIRT1-dependent and SIRT1-independent function in mediating a salutary effect following HI.

### B. Dichloroacetate (DCA)

Dichloroacetate (DCA) is an orphan drug previously used for lactic acidosis and MELAS (mitochondrial cytopathies). DCA is a potent inhibitor of the mitochondrial enzyme pyruvate dehydrogenase kinase (Pdk). Activation of Pdk leads to inhibition of mitochondrial gatekeeper, pyruvate dehydrogenase (PDH) that converts pyruvate to acetyl CoA. Inhibition of Pdk by DCA results in activation of PDH, increased turnover of pyruvate to acetyl CoA and increased generation of ATP. During hypoxic condition or in aerobic glycolysis, inhibition of Pdh by Pdk results in a shift in energy production from mitochondria to glycolysis DCA treatments restore the activity of Pdh leading to mitochondrial functional improvement.

### C. Resveratrol

Resveratrol (3,5,4-tribydroxystilbene) (RSV) is a phytoalexin produced in several species of plants including grapevines (Vitis vinifera) (Poulose, N., et al., Biochimica et biophysica acta, 1852(11):2442-2455 (2015)). Red wine contains a high level of resveratrol and it is reported to be one of the main factors contributing to the French Paradox. The term “French Paradox” was coined to describe the inverse relation between coronary heart disease mortality and the predominantly red wine consumption seen in France (Renaud, S., et al., Lancet, 339(8808):1523-1526 (1992)). Resveratrol has been shown to increase lifespan (Park, S. J., et al., Cell, 148(3):421-433 (2012); Bhullar, K. S., et al., Biochimica et biophysica acta, 1852(6):1209-1218 (2015)), decrease vascular inflammation, confer vasoprotection and reduce myocardial I/R injury (Lekli, I., et al., Am J Physiol Heart Circ Physiol, 294(2):H859-866 (2008); Deng, Z. Y., et al., Inflamm Res, 64(5):321-332 (2015)) and is antioxidant and anti-inflammatory (Xu, W., et al., Clin Exp Pharmacol Physiol, 42(10):1075-1083 (2015); Csiszar, A., et al., Am J Physiol Heart Circ Physiol, 291(4):H1694-1699 (2006)). Resveratrol is also an allosteric activator of SIRT1. The deregulation of mitochondrial function is a known consequence of injury and resveratrol is known to modulate mitochondrial function in ischemic damage, metabolic diseases and aging (Price, N. L., et al., Cell Metabolism, 15(5):675-690 (2012); Gomes, A. P., et al., Cell, 155(7):1624-1638 (2013); Kozlov, A. V., et al., Ann Intensive Care, 1(1):41 (2011); Zhang, Q., et al., Shock, 34(1):55-59 (2010)).

### III. Formulations and Administration

### A. Pharmaceutically Acceptable Salts

Resveratrol, dichloroacetate, and niacin (collectively referred to as “the compounds”) may be administered as one pharmaceutically acceptable composition or as individual pharmaceutically acceptable compositions. In one embodiment, the compounds are formulated as pharmaceutically acceptable salts. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds which are generally prepared by reacting the free base with a suitable organic or inorganic acid. Representative salts of basic compounds of the present invention include, but are not limited to, the following: acetate, ascorbate, adipate, alginate, aspirate, benzenesulfonate, benzoate, bicarbonate, bisulfate, bitartrate, borate, bromide, butyrate, camphorate, camphorsulfonate, camsylate, carbonate, chloride, clavulanate, citrate, cyclopentane propionate, diethylacetic, digluconate, dihydrochloride, dodecylsulfanate, edetate, edisylate, estolate, esylate, ethanesulfonate, formic, fumarate, gluceptate, glucoheptanoate, gluconate, glutamate, glycerophosphate, glycollylarsanilate, hemisulfate, heptanoate, hexanoate, hexylresorcinate, hydrabamine, hydrobromide, hydrochloride, 2-hydroxyethanesulfonate, hydroxynaphthoate, iodide, isonicotinic, isothionate, lactate, lactobionate, laurate, malate, maleate, mandelate, mesylate, methylbromide, methylnitrate, methylsulfate, methanesulfonate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, phosphate/diphosphate, pimelic, phenylpropionic, polygalacturonate, propionate, salicylate, stearate, sulfate, subacetate, succinate, tannate, tartrate, teoclate, thiocyanate, tosylate, triethiodide, trifluoroacetate, undeconate, valerate and the like. Furthermore, where the compounds carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, dicyclohexyl amines and basic ion-exchange resins, such as arginine, betaine, caffeine, choline, N,N-dibenzylethylenediamine, diethylamine, 2-diethylaminoethanol, 2-dimethylaminoethanol, ethanolamine, ethylamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Also, included are the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

These salts can be obtained by known methods, for example, by mixing the compounds with an equivalent amount and a solution containing a desired acid, base, or the like, and then collecting the desired salt by filtering the salt or distilling off the solvent. The compounds and salts thereof may form solvates with a solvent such as water, ethanol, or glycerol. The compounds may form an acid addition salt and a salt with a base at the same time according to the type of substituent of the side chain.

### B. Stereoisomeric Forms

All stereoisomeric forms of the compounds can be used. Centers of asymmetry that are present in the compounds can all independently of one another have (R) configuration or (S) configuration. When bonds to the chiral carbon are depicted as straight lines in the structural Formulas of the invention, it is understood that both the (R) and (S) configurations of the chiral carbon, and hence both enantiomers and mixtures thereof, are embraced within the Formula. Similarly, when a compound name is recited without a chiral designation for a chiral carbon, it is understood that both the (R) and (S) configurations of the chiral carbon, and hence individual enantiomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of the compounds or it can be done on a final racemic product. Absolute stereochemistry may be determined by X-ray crystallography of crystalline products or crystalline intermediates which are derivatized, if necessary, with a reagent containing a stereogenic center of known configuration. Where the compounds are capable of tautomerization, all individual tautomers as well as mixtures thereof are included in the scope of this invention. The present invention includes all such isomers, as well as salts, solvates (including hydrates) and solvated salts of such racemates, enantiomers, diastereomers and tautomers and mixtures thereof.

### C. Substituents

It is understood that substituents and substitution patterns on the compounds can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted” (with one or more substituents) should be understood as meaning that the group in question is either unsubstituted or may be substituted with one or more substituents.

### D. Amorphous and Crystalline Forms

Furthermore, compounds may exist in amorphous form and/or one or more crystalline forms, and as such all amorphous and crystalline forms and mixtures thereof of the compounds of Formula I are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms.

### E. Prodrugs

Any pharmaceutically acceptable pro-drug modification of the compounds which results in conversion in vivo to a compound within the scope of this invention is also within the scope of this invention. For example, esters can optionally be made by esterification of an available carboxylic acid group or by formation of an ester on an available hydroxy group in a compound. Similarly, labile amides can be made. Pharmaceutically acceptable esters or amides of the compounds of this invention may be prepared to act as pro-drugs which can be hydrolyzed back to an acid (or —COO— depending on the pH of the fluid or tissue where conversion takes place) or hydroxy form particularly in vivo and as such are encompassed within the scope of this invention.

Accordingly, the compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof, pro-drug forms thereof, and salts of pro-drug forms thereof, where such forms are possible unless specified otherwise.

### F. Preparation Forms

Suitable solid or galenical preparation forms are, for example, granules, powders, coated tablets, tablets, (micro)capsules, suppositories, syrups, juices, suspensions, emulsions, drops or injectable solutions and preparations having prolonged release of active substance, in whose preparation customary excipients such as vehicles, disintegrants, binders, coating agents, swelling agents, glidants or lubricants, flavorings, sweeteners and solubilizers are used. Frequently used auxiliaries which may be mentioned are magnesium carbonate, titanium dioxide, lactose, mannitol and other sugars, talc, lactose, gelatin, starch, cellulose and its derivatives, animal and plant oils such as cod liver oil, sunflower, peanut or sesame oil, polyethylene glycol and solvents such as, for example, sterile water and mono- or polyhydric alcohols such as glycerol.

### H. Dosage Regimen

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the compounds, when used for the indicated effects, will range between about 0.01 mg per kg of body weight per day (mg/kg/day) to about 30 mg/kg/day, preferably 0.025-7.5 mg/kg/day, more preferably 0.1-2.5 mg/kg/day, and most preferably 0.1-0.5 mg/kg/day (unless specified otherwise, amounts of active ingredients are on free base basis). For example, an 80 kg patient would receive between about 0.8 mg/day and 2.4 g/day, preferably 2-600 mg/day, more preferably 8-200 mg/day, and most preferably 8-40 mg/kg/day. A suitably prepared medicament for once a day administration would thus contain between 0.8 mg and 2.4 g, preferably between 2 mg and 600 mg, more preferably between 8 mg and 200 mg, and most preferably 8 mg and 40 mg, e.g., 8 mg, 10 mg, 20 mg and 40 mg. One embodiment provides an oral formation containing 200 to 600 mg of the active agents.

Advantageously, the pharmaceutical compositions may be administered in divided doses of two, three, or four times daily. For administration twice a day, a suitably prepared medicament would contain between 0.4 mg and 4 g, preferably between 1 mg and 300 mg, more preferably between 4 mg and 100 mg, and most preferably 4 mg and 20 mg, e.g., 4 mg, 5 mg, 10 mg and 20 mg. Another embodiment provides a sublingual formulation containing deliver between 0.025-7.5 mg/kg/day, preferably 0.1-2.5 mg/kg/day, and more preferably 0.1-1.0 mg/kg/day of each active agent.

Another embodiment provides a sublingual formulation containing between 0.4 mg and 4 g, preferably between 1 mg and 300 mg, more preferably between 4 mg and 100 mg, and most preferably 4 mg and 20 mg, e.g., 4 mg, 5 mg, 10 mg and 20 mg of each active agent.

Intravenously, the patient would receive the active ingredient in quantities sufficient to deliver between 0.025-7.5 mg/kg/day, preferably 0.1-2.5 mg/kg/day, and more preferably 0.1-1.0 mg/kg/day. Such quantities may be administered in a number of suitable ways, e.g. large volumes of low concentrations of active ingredient during one extended period of time or several times a day, low volumes of high concentrations of active ingredient during a short period of time, e.g. once a day. Typically, a conventional intravenous formulation may be prepared which contains a concentration of active ingredient of between about 0.01-1.0 mg/ml, e.g. 0.1 mg/ml, 0.3 mg/ml, and 0.6 mg/ml, and administered in amounts per day of between 0.01 ml/kg patient weight and 10.0 ml/kg patient weight, e.g. 0.1 ml/kg, 0.2 ml/kg, 0.5 ml/kg. In one example, an 80 kg patient, receiving 8 ml twice a day of an intravenous formulation having a concentration of active ingredient of 0.5 mg/ml, receives 8 mg of active ingredient per day. Glucuronic acid, L-lactic acid, acetic acid, citric acid or any pharmaceutically acceptable acid/conjugate base with reasonable buffering capacity in the pH range acceptable for intravenous administration may be used as buffers. The choice of appropriate buffer and pH of a formulation, depending on solubility of the drug to be administered, is readily made by a person having ordinary skill in the art.

The pharmaceutical compositions can be administered both as a monotherapy and in combination with other therapeutic agents, including coagulants and antiarrhythmics.

The pharmaceutical compositions are preferably administered alone to a mammal in a therapeutically effective amount. However, the compounds also be administered in combination or alternation with each other or with an additional therapeutic agent, as defined below, to a mammal in a therapeutically effective amount. When administered in a combination, the combination of compounds in preferably, but not necessarily, a synergistic combination. Synergy, as described for example by Chou and Talalay, Adv. Enzyme Regul. 1984, 22, 27-55, occurs when the effect (in this case, inhibition of the desired target) of the compounds when administered in combination is greater than the additive effect of the compounds when administered alone as a single agent or when less than of each compound is needed than when administered alone. In general, a synergistic effect is most clearly demonstrated at suboptimal concentrations of the compounds. Synergy can be in terms of lower cytotoxicity, increased physiological effect, or some other beneficial effect of the combination compared with the individual components.

By “administered in combination” or “combination therapy” it is meant that the compounds and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

### III. Methods of Treatment

The combination of resveratrol, dichloroacetate, and niacin can be used to improve organ function in subjects that have hemorrhagic injury, for example battle wounds. One embodiment provides a method for treating hemorrhagic shock in a subject in need thereof by administering to the subject an effective amount of niacin, dichloroacetate, and resveratrol to improve organ function relative to an untreated control. Organ function include one or more vital organs including liver, lungs, intestine, kidney, heart. The niacin, dichloroacetate, and resveratrol can be administered in combination as a pharmaceutical composition or each can be administered separately either simultaneously, in alternation, or in succession. In one embodiment, each of the niacin, dichloroacetate, and resveratrol are administered at 1 mg/kg to 10 mg/kg, preferably about 2 mg/kg.

In another embodiment, the niacin, dichloroacetate, and resveratrol are administered to a subject having hemorrhagic injury without administering resuscitation fluids.

Another embodiment provides a method for treating hemorrhagic injury in a subject in need thereof comprising administering to the subject the pharmaceutical composition containing an effective amount of niacin, dichloroacetate, and resveratrol to reduce mortality associated with hemorrhagic injury or increase survivability.

Yet another embodiment provides a method for reducing the incidence of death due to hemorrhagic injury by administering to a subject in need thereof an effective amount of niacin, dichloroacetate, and resveratrol.

### EXAMPLES

### Example 1

### Resveratrol Improves Cardiac Function Following Severe Hemorrhage In A Rat Model

### Materials and Methods

### Animals

Male Sprague Dawley rats (250-350 g) were obtained from Charles River Laboratory (Wilmington, Mass., USA). The experiments performed in this study including surgical procedures were approved by the Institutional Animal Care and Use Committee (IACUC) at Augusta University. All animals were housed in Augusta University animal facility during the experiments.

### Hemorrhage Procedure

The animals were fasted overnight (water allowed ad libitum) prior to the sham or hemorrhagic injury (HI) procedure. Hemorrhagic procedure was as described before with some modification (Jian, B. et al., Mol. Med., 18:209-14 (2012). The animals were anesthetized with 2.5% isoflurane (Henry Schein, Dublin, Ohio, USA) and were placed on Plexiglas plate in a supine position. Isoflurane was administered in oxygen using an anesthetic vaporizer. Midline laparotomy (5 cm) was performed in both sham and HI animals to induce soft tissue trauma. Incision was closed aseptically in two layers with sutures. Two femoral arteries and one femoral vein were cannulated (PE-50 tubing), one artery was hooked up to a Blood Pressure Analyzer (Digi-Med; Micro-Med Inc., Louisville, Ky., USA) to monitor mean arterial pressure (MAP) and bleeding was performed through the other artery. Femoral vein was cannulated to administer fluids. Surgical sites were bathed with bupivacaine. Sham animals were not subjected to bleeding or resuscitation. Upon awakening, the animals in HI groups were bled rapidly within the first 10 min to a MAP of 40±5 mmHg. The bleeding was continued for 45 min, maintaining the low MAP, until 60% of circulatory blood volume was withdrawn. The animals were maintained in the state of shock by maintaining MAP at 40±5 for another 45 min. During this time not more than 40% of the shed blood volume in the form of Ringer lactate was given to maintain the blood pressure. Resuscitation was performed with Ringer lactate for one hour on animals in the respective groups. Vehicle (dimethyl sulfoxide [DMSO]; 120 μL/mouse) or drug was administered at 10 min from the onset of resuscitation or immediately after shock period when there was no resuscitation.

### Results

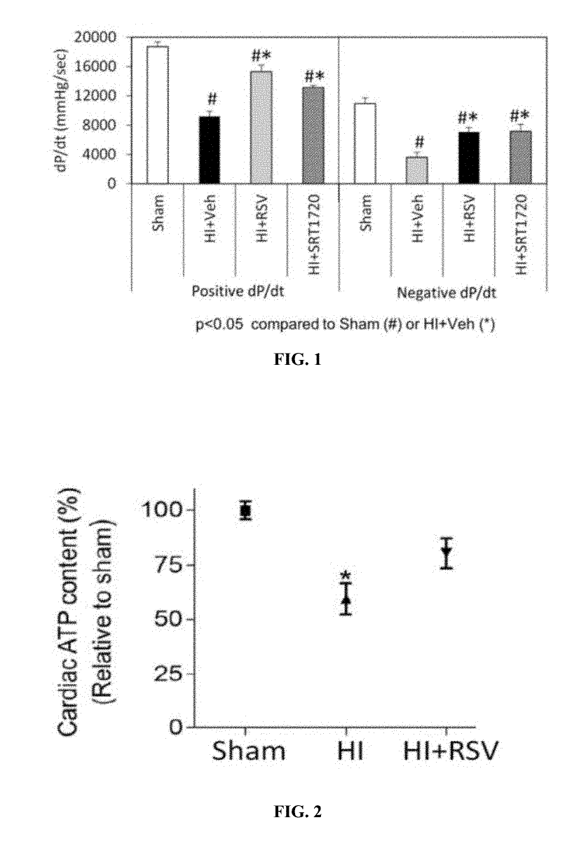

FIG. 1 shows that +dp/dt and −dp/dt (not shown) were significantly reduced following HI and restored with resveratrol (RSV) treatment; n=4-6, p<0.05 vs sham.

### Example 2

### Mitochondrial Function Following HI

### Materials and Methods

ATP content was measured in left ventricular tissue from animals in each group (sham, HI, HI−RSV) by a bioluminescence assay (ATP determination kit; Invitrogen). n=4-6, bars: mean+SEM. *p<0.05.

### Results

One of the master regulators of mitochondrial function is Pgc-1α, the expression of which is declined following HI (Jian, B., et al., Molecular Medicine, 18:209-214 (2012); Hsieh, Y. C., et al., FASEB Journal, 20(8):1109-1117 (2006); Wu, Z., et al., Cell, 98(1):115-124 (1999)). FIG. 2 shows declined mitochondrial function following HI as evidenced by decreased tissue ATP, decreased mitochondrial complex I activity and increased cytosolic cytochrome c following HI.

### Example 3

### Resveratrol Improves Survival after HI

### Materials and Methods

Rats were subjected to HI administered Veh (n=12) and HI+RSV (n=8) groups with a dose of 10 mg/kg body weight.

### Results

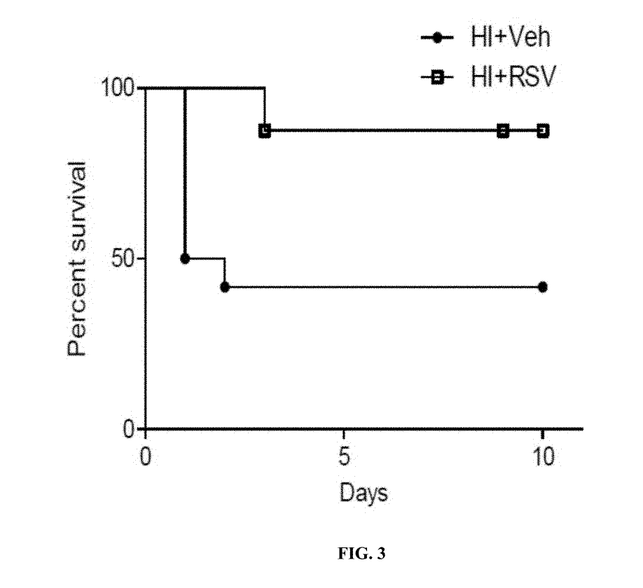

As seen in FIG. 3, in the 10-day survival study, most of the mortality seen in untreated animals was within a day of hemorrhagic procedure and the results further show that early intervention with resveratrol can reduce mortality significantly. The mean weight gain was also significantly improved in animals that received resveratrol (data not shown).

### Example 4

### Resveratrol and SRT1720 Prolong Life After HI Without Resuscitation

### Materials and Methods

Rats were subjected to HI and Veh, RSV or SRT1720 administered (n=5-6). (RSV: 10 mg/Kg body weight; SRT1720: 2 mg/Kg body weight.

### Results

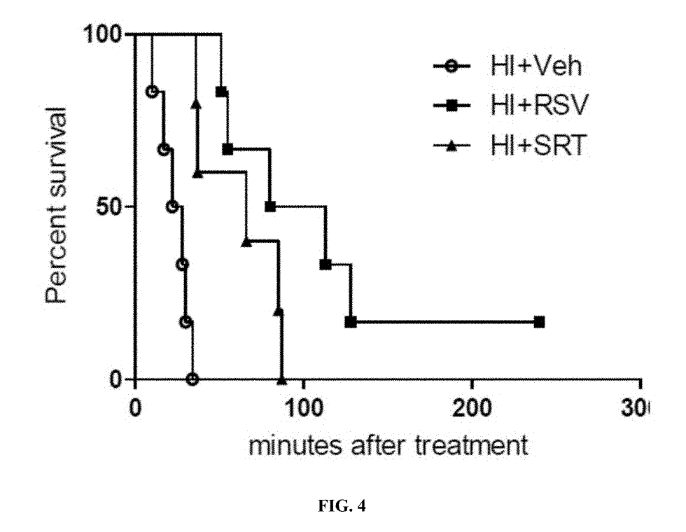

The data show that (1) significant mortality following HI in spite of resuscitation is observed within 24 hours (FIG. 3), (2) the mortality is significantly reduced when resveratrol is administered along with resuscitation fluid (FIG. 3), (3) almost all rats die within an hour in the absence of resuscitation fluid (FIG. 4) and (4) when resveratrol is administered following HI in the absence of resuscitation, the animals survived for a significantly longer period of time (FIG. 4).

### Example 5

### Dichloroacetate (DCA) Prolongs Life After HI Without Resuscitation

### Materials and Methods

Rats were divided into the following treatment groups HI+Veh (n=7), HI+DCA 1 mg/kg (n=3), HI+DCA 10 mg/kg (n=5), HI+DCA 25 mg/kg (n=5).

### Results

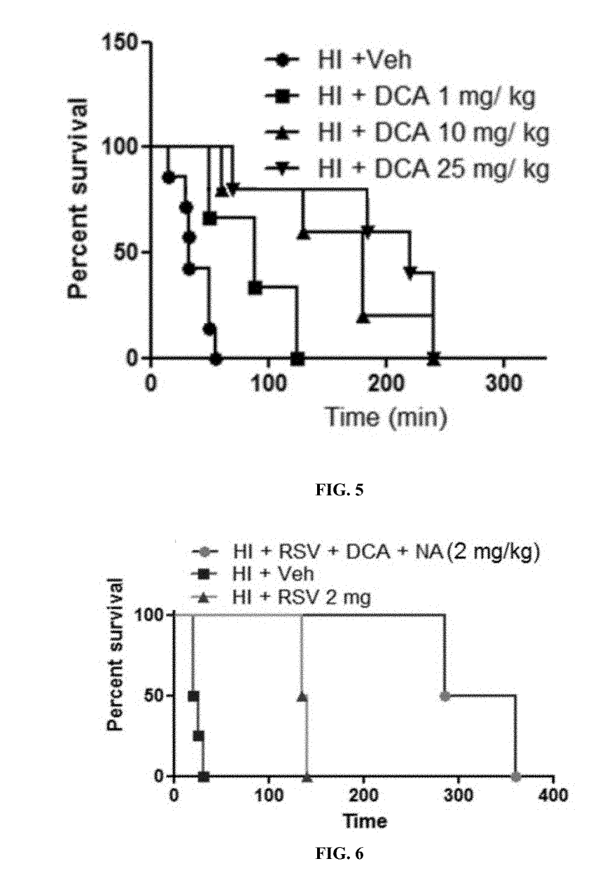

In order to further confirm the role of mitochondria in HI, the effect of dichloroacetate (DCA), a classic inhibitor of Pdk, was tested in the rat model of hemorrhagic shock and a significant salutary effect was observed (FIG. 5). Pdk1 phosphorylates and inhibit pyruvate dehydrogenase (PDH), a mitochondrial gatekeeper, resulting in decreased conversion of pyruvate to acetyl CoA, decreased mitochondrial respiration (Jian, B., et al., Molecular Medicine, 20:10-16 (2014); Granot, H., et al., Circ Shock, 15(3):163-173 (1985); Ruggieri, V., et al. Oncotarget, 6(2):1217-1230 (2015)). The inhibition of Pdk by DCA enhanced mitochondrial function and improved survival in association with enhancement of aerobic metabolism.

### Example 6

### Nicotinic Acid (Niacin) Prolongs Life After HI Without Resuscitation

### Materials and Methods

Rats were administered HI+Veh (n=3), HI+Niacin 10 mg/kg (n=6), HI+Niacin 5 mg/kg (n=6) and HI+Niacin 1 mg/kg (n=4). p<0.05 versus HI+Veh, all curves.

### Results

FIG. 6 shows prolonged survival when niacin was administered following HI in the rats. Niacin metabolizes to produce NAD+, which is a cofactor for SIRT1, a target of resveratrol, that enhances mitochondrial function. Niacin exerts anti-inflammatory effect by binding to its cognate receptor, GPR109a.

### Example 7

### Resveratrol (RSV)+Dichloroacetate (DCA) and Niacin (NA) Prolongs Life After HI in the Absence of Resuscitation

### Materials and Methods

Rats were administered HI+Veh (n=4), HI+RSV+DCA+NA 2 mg/Kg (n=2) and HI+RSV 2 mg/Kg (n=2).

### Results

The pathways triggered by resveratrol, niacin and DCA converge on mitochondria, nevertheless, resveratrol and niacin also exert salutary effect through other pathways such as that involved in NF-kb mediated inflammatory response. Collectively, the data show that HI leads to a disturbance of metabolic networks, and methods to restore the biologic coordinates of metabolic checkpoints converging on mitochondria can reduce organ dysfunction and mortality following HI. Based upon these results a combinatorial formulation of resveratrol, niacin and DCA is more effective in the treatment of HI, as this (1) reduces the dose of individual drug components and (2) maintain more uniform drug response across genetic variations as compared to single drug therapies. In initial experiments 2 mg/Kg resveratrol alone or a combination formulation of resveratrol, niacin and DCA (2 mg/kg each) without fluid resuscitation was administered (FIG. 7). The combinatorial treatment effectively reduced the dose of each of the components to 2 mg/Kg each a completely unexpected result.

I claim:

1. A pharmaceutical composition comprising an effective amount of niacin, dichloroacetate, and resveratrol or pharmaceutically acceptable salts, solvates, or enantiomers thereof to improve organ function relative to an untreated control following severe hemorrhage in the subject.

2. The pharmaceutical composition of claim 1, wherein the pharmaceutical composition is formulated as tablet.

3. The pharmaceutical composition of claim 1, wherein the niacin, dichloroacetate, and resveratrol are each present in about 1 to 10 mg/kg.

4. The pharmaceutical composition of claim 3, wherein the niacin, dichloroacetate, and resveratrol are each present in about 2 mg/kg

5. A method for treating hemorrhagic stroke in a subject in need thereof comprising administering to the subject an effective amount of niacin, dichloroacetate, and resveratrol to improve cardiac function relative to a control.

6. The method of claim 5, wherein the niacin, dichloroacetate, and resveratrol are administered in combination.

7. The method of claim 5, wherein the niacin, dichloroacetate, and resveratrol are administered successively or in alternation.

8. A method for treating hemorrhagic stroke in a subject in need thereof comprising administering to the subject the pharmaceutical composition of claim 1.

9. The method of claim 5, wherein resuscitation fluids are not administered to the subject.

10. A method for increasing survival from hemorrhagic shock in a subject in need thereof, comprising administering to the subject any one of pharmaceutical compositions of claim 1.

11. The method of claim 10, wherein the pharmaceutical compositions are administered to the subject in the absence of resuscitation fluids.

12. A method for reducing mortality due to hemorrhagic shock in a subject in need thereof, comprising administering to the subject the pharmaceutical composition of claim 1 in the absence of administering resuscitation fluids.

13. A method of treating hemorrhagic injury in a subject in need thereof consisting of administering to the subject 2 mg/kg resveratrol, 2 mg/kg niacin, and 2 mg/kg dichloroacetate in combination, alternation, or succession to improve cardiac function in the subject relative to an untreated control.

14. A pharmaceutical composition comprising 2 mg/kg resveratrol, 2 mg/kg niacin, and 2 mg/kg dichloroacetate, and optionally a pharmaceutically acceptable excipient.

15. A pharmaceutical composition consisting of 2 mg/kg resveratrol, 2 mg/kg niacin, and 2 mg/kg dichloroacetate, and a pharmaceutically acceptable excipient.
